(19) United States
(12) Patent Application Publication
(10) Pub. No.: US 2003/0118633 A1
(43) Pub. Date: Jun. 26, 2003

(54) Combination therapy

(76) Inventor: Ebrahim Versi, Gladstone, NJ (US)

Correspondence Address: DINSMORE & SHOHL, LLP, 1900 CHEMED CENTER, 255 EAST FIFTH STREET, CINCINNATI, OH, 45202, US

(21) Appl. No.: 10/289,903

(22) Filed: Nov. 7, 2002

Related U.S. Application Data
(60) Provisional application No. 60/344,507, filed on Nov. 9, 2001.

(51) Int. Cl. A61K 31/137; A61K 9/70; A61K 9/48; A61K 9/20

(52) U.S. Cl. 424/449; 514/649; 424/465; 424/451

(57) ABSTRACT

The invention relates to a method of treating unstable or overactive urinary bladder, wherein the method comprises administering to a patient in need of such treatment an antimuscarinic agent in a pharmaceutically effective amount thereof and estrogen agonist in a pharmaceutically effective amount thereof. The antimuscarinic agent is preferably tolterodine or a tolterodine-related compound. The invention also relates to a pharmaceutical formulation containing an antimuscarinic agent, estrogen agonist and pharmaceutically excipients and the use of the combination for the manufacture of a therapeutical formulation for treating unstable or overactive urinary bladder.

The present invention relates to an improved method of treating unstable or overactive urinary bladder wherein the method comprises administering to a patient in need of such treatment an antimuscarinic agent in a pharmaceutically effective amount thereof and estrogen agonist in a pharmaceutically effective amount thereof. A therapeutical formulation thereof is also claimed.

### BACKGROUND

A substantial part (5-10%) of the adult population suffers from urinary incontinence and the prevalence, particularly of so-called urge incontinence, increases with age. The symptoms of an unstable or overactive bladder comprise urge incontinence, urgency and urinary frequency and others. It is assumed that unstable or overactive bladder is caused by uncontrolled contractions of the bundles of smooth muscle fibres forming the muscular coat of the urinary bladder (the detrusor muscle) during the filling phase of the bladder. These contractions are mainly controlled by cholinergic muscarinic receptors, and the pharmacological treatment of unstable or overactive bladder has been based on muscarinic receptor antagonists.

A commercial available drug has for a long time been oxybutynin.

An improved muscarinic receptor antagonist, tolterodine. (R)-N,N-diisopropyl-3-(2-hydroxy -5-methylphenyl)-3-phenylpropanamine, has been marketed for the treatment of urge incontinence and other symptoms of unstable or overactive urinary bladder.

Both tolterodine and its major, active metabolite, the 5-hydroxymethyl derivative of tolterodine, which significantly contributes to the therapeutic effect, have considerably less side effects than oxybutynin, especially regarding the propensity to cause dry mouth. The selective effect of tolterodine in humans is described in Stahl, M. M. S., et al., Neurourology and Urodynamics 14 (1995) 647-655, and Bryne, N., International Journal of Clinical Pharmacology and Therapeutics, Vol. 35, No. 7 (1995) 287-295.

The currently marketed administration form of tolterodine is either a tablet containing 1 mg or 2 mg of tolterodine L-tartrate for immediate release in the gastrointestinal tract or a controlled release tablet. The side effects, such as dry mouth, are much lower than for oxybutynin.

Tolterodine, its corresponding (S)-enantiomer and racemate and the preparation thereof are described in e.g. WO 89/06644. For a description of the active (R)-5-hydroxymethyl metabolite of tolterodine (as well as the (S)-5-hydroxymethyl metabolite), it may be referred to WO 94/11337. The (S)-enantiomer and its use in the treatment of urinary and gastrointestinal disorders is described in WO 98/03067.

A controlled release tablet is described in WO00/27364 and WO0012069.

WO98/43942 discloses therapeutically active diarylpropylamines which have favourable anticholinergic properties, and which can be used for for the treatment of urinary incontinence related disorders.

An article by Goode P S et al in American Journal of the Medical Sciences, (October 1997 ) 314 (4) 262-7 “Pharmacologic treatment of lower urinary tract dysfunction in geriatric patient” gives an overview over treatment of lower urinary tract dysfunction in geriatric patients with different medicaments. As anticholinergic medicaments which could be used against urinary frequency and/or urge incontinence are mentioned oxybutynin, hyoscyamine, and dicyclomine. Phenylpropanolamine are mentioned to be useful against stress urinary incontinence, It is stated in the abstract: “In addition to these medications (alpha adrenergic agonist, my comment) in postmenopausal women, estrogen seems to have (emphasis added) an additive effect for both urge and stress incontinence.” No specific combination or data are, however, given in this article and the combination is only a hypothesis for estrogen in combination with oxybutynin..

In the article “Management of coexistent Stress and Urge urinary Incontinence” by M Karram et al, Obstetric & Gynecology, Vol 73 No 1, January 1989, women were treated with oxybutynin and estrogen, imipramine and estrogen and oxybutynin, imipramine and estrogen, respectively. It was found that the combination of oxybutynin and imipramine was the most successful. No conclusion can be drawn from this article about the synergistic effect of the combination of an antimuscarinic agent such as tolterodine and estrogen.

U.S. Pat. No. 6,262,115 discloses the use of oxybutunin and estrogens in management of incontinence and hormone replacement, but there is no disclosure of a synergistic effect of the two drugs in the treatment of unstable or overactive urinary bladder.

Olsson B et al, Clinial Therap Vol 23. Nr 11, 2001, p. 1876-1888 discloses the use of tolterodine and ethinyl estradiol as contraceptive. The results showed that there were no relevant interaction with ethinylestradiol and tolterodine on the contraceptive effect.

### THE INVENTION

According to the present invention it has now surprisingly been found that the combination of an antimuscarinic agent and estrogen agonist, especially estrogen, gives an effect in the treatment of various incontinence problems. As anti-muscarinic agents can be mentioned tolterodine, fesoterodine, oxybutynin, S-oxybutynin, darifenacin, hyoscyamine, dicyclomine, oxytrol, solifenacin, propiverin, temiverine and trospium and ipratropium. Tolrerodine has an effect on the sensory axis and so does estrogen and tolterodine and its metabolite is therefore the preferred drug.

The administration can be simultaneous, separat or sequential.

In one aspect, the present invention therefore a method of treating unstable or overactive urinary bladder, which method comprises administering to a patient in need of such treatment, especially a mammal, an antimuscarinic agent, especially tolterodine or a tolterodine-related compound, or a pharmaceutically acceptable salt thereof and estrogen agonist, especially estrogen, by any administration method.

In another aspect, the present invention provides a pharmaceutical formulation containing an antimuscarinic agent e.g. tolterodine or a tolterodine-related compound and estrogen.

Still another aspect of the present invention provides the use of an antimuscarinic agent e.g. tolterodine or a tolterodine-related compound, or a pharmaceutically acceptable salt thereof together with estrogen, for the manufacture of a therapeutical formulation for treating unstable or overactive urinary bladder.

The two drugs can be given either together in the same composition or as different formulation e.g. orally or rectally or vaginally. They can be given at the same time or sequencetly. The oral formulation can be e.g. as controlled release forms or as buccal tablets. The formulation of each drug can be e g. as rectal suppositories, subcutaneous implants, formulations for intramuscular administration or vaginally.

The preferred adiministered amount of the antimuscarininc agent is from about 0.05 mg to abut 12 mg, more preferred amount is from about 0.1 mg to about 6 mg and most preferable about 0.2 to about 5 mg, but is depending on which drug is used.

The patient is preferably a menopause woman.

Overactive urinary bladder encompasses variant of urinary disorders including overactive detrusor (detrusor instability, detrusor hyperreflexia) and sensory urgency and the symptoms of detrusor overactivity, e.g. urge incontinence, urgency and urinary frequency and LUTS (Lower urinary Tract Symptoms including obstructive urinary symptoms such as slow urination, dribbling at the end of urination, inability to urinate and/or the need to strain to urinate at an acceptable rate or irritate symptoms such as frequency and/or urgency).

Also other conditions are included, which give rise to urinary frequency, urgency and/or urge incontinence. Overactive bladder disorders also include nocturia and mixed incontinence. While overactive bladder is often associated with detrusor muscle instability, disorders of bladder function may also be due to neuropathy of the central nervous system (detrusor hyperreflexia) including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis. A specific problem which can be treated by the claimed method is a dry overactive bladder, which includes frequency, urgency and nocturia.

As mentioned above, the chemical name of tolterodine is (R)-N,N-diisopropyl-3-(2-hydroxy-5-methylphenyl)-3-phenylpropanamine. The term “tolterodine-related compound” is meant to encompass the major, active metabolite of tolterodine, i.e. (R)-N,N-diisopropyl-3-(2-hydroxy-5-hydroxymethylphenyl)-3-phenylpropanamine; the corresponding (S)-enantiomer to tolterodine, i.e. (S)-N,N-diisopropyl-3-(2-hydroxy-5-methylphenyl)-3-phenylpropanamine: the 5-hydroxymethyl metabolite of the (S)-enanitiomer, i.e. (S)-N,N-diisopropyl-3-(2-hydroxy-5-hydroxymethylphenyl )-3-phenylpropanamine; as well as the corresponding racemate to tolterodine, i.e. (R,S)-N,N-diisopropyl-3-(2-hydroxy-5-methylphenyl)-3-phenylpropanamine; and prodrug forms thereof.

By the term “active moiety or moities” is meant the sum of free or unbound (i.e. not protein bound) concentrations of (i) tolterodine and active metabolite thereof, when tolterodine (or prodrug form) is administered; or (ii) tolterodine and active metabolite thereof and/or (S)-enantiomer to tolterodine and active metabolite thereof, when the corresponding racemate (or prodrug form) is administered: or (iii) active metabolite, when the (R)-5-hydroxymethyl metabolite of tolterodine (or prodrug form) is administered; or (iv) (S)-enantiomer to tolterodine and active metabolite thereof, when the (S)-enantiomer (or prodrug) is administered, or (v) active (S)-metabolite, when the (S) 5-hydroxymethyl metabolite is administered.

As estrogen agonists the compounds estradiol, estriol, estrone and dienoestrone, which could be conjugated or esterified, are suggested.

Different products containing estrogen can be used, such as a tablet for oral use, cream or blaster for transdermal use or devices for intravaginal use e.g. Oestring®, which is a an intravaginal device comprising a combination of 17 .beta.-estradiol and a supporting matrix for treating hypoestrogenic women and described in U.S. Pat. No. 4,871,543.

Other preferred products are Vagifem® and Activelle®.

### EXAMPLE 1

Menopause women with overactive bladder, are treated during 3 months with the following regime:

Intravaginal estrogen, Vagifem® 25 μg daily and tolterodine, 4 mg once daily.

Four groups are studied:

I. Combination of Vagifem® 25 μg daily and tolterodine, 4 mg once daily during three months.

II. Vagifem 25 μg daily during three months.

III. Tolterodine, 4 mg once daily during three months.

IV. The same regime as group I but placebo medicaments.

The patients are followed and urinary dairy are to be kept and the patient perseption are studied.

The synergetic effect on the overactive bladder is registered.

### EXAMPLE 2

Menopause women with overactive bladder, are treated during 3 months with the following regime:

2 mg oral estrogen daily and tolterodine, 4 mg once daily.

Foul groups are studied:

I. Combination of 2 mg oral estrogen daily and tolterodine, 4 mg once daily during three months.

II. 2 mg oral estrogen daily during three months.

III. Tolterodine, 4 mg once daily during three months.

IV. The same regime as group I but placebo medicaments.

The patients are followed and urinary dairy are to be kept and the patient perseption are studied.

The synergetic effect on the overactive bladder is registrered.

### EXAMPLE 3

Menopause women with overactive bladder, are treated during 3 months with the following regime:

1 mg oral Activella® (1 mg edstradiol and 0.5 mg norethidrone) daily and tolterodine, 4 mg once daily.

Four groups are studied:

I Combination of 1 mg oral Activella® daily and tolterodine, 4 mg once daily during three months.

II 1 mg oral Activella® daily during three months.

III Tolterodine, 4 mg once daily during three months.

IV The same regime as group I but placebo medicaments.

The patients are followed and urinary dairy are to be kept and the patient perseption are studied.

The synergetic effect on the overactive bladder is registrered.

1. A method of treating unstable or overactive urinary bladder, wherein the method comprises administering to a patient in need of such treatment an antimuscarinic agent in a pharmaceutically effective amount thereof and estrogen agonist in a pharmaceutically effective amount thereof.

2. A method according to claim 1 in which the antimuscarinic agent and estrogen agonist are given simultaneously, separatly or sequentially.

3. A method according to claim 1 or 2 in which the antimuscarinic agent is tolterodine or a tolterodine-related compound, or a pharmaceutically acceptable salt thereof.

4. The method according to any one of claims 1 to 3, wherein the antimuscarinic agent drug is tolterodine.

5. The method according to claim 4, wherein tolterodine is administered in an amount of 0.1 to 12 mg daily.

6. The method according to any one of claims 1 to 5, wherein estrogen agonist is given orally.

7. The method according to any one of claims 1 to 5, wherein estrogen agonist is given vaginally.

8. A pharmaceutical formulation containing an antimuscarinic agent in a pharmaceutically effective amount thereof and estrogen agonist in a pharmaceutically effective amount thereof and pharmaceutically excipients.

9. A pharmaceutical formulation accordig to claim 8 for simultaneous, separat or sequential administration of the antimuscarinic agent and estrogen agonist.

10. A pharmaceutical formulation according to claim 8 or 9 in which the antimuscarinic agent is tolterodine or a tolterodine-related compound.

11. A pharmaceutical formulation according to claim 10 in which the antimuscarinic agent is tolterodine.

12. A pharmaceutical formulation according to claim 8, wherein the antimuscarinic agent is present in an amount of 0.1 to 12 mg.

13. The formulation according to any one of claims 8-12, which is a capsule or tablet for oral administration once daily.

14. The formulation according to any one of claims 8-12, which is a transdermal preparation, preferably a transdermal patch.

15. The formulation according to any one of claims 10 to 13, which provides controlled release of tolterodine.

16. Use of an antimuscarinic agent in a pharmaceutically effective amount thereof and estrogen agonist in a pharmaceutically effective amount thereof for the manufacture of a therapeutical formulation for treating unstable or overactive urinary bladder.

17. Use according to claim 15 for simultaneous, separat or sequential administration.

18. Use according to claim 15 in which the antimuscarinic agent is tolterodine or a tolterodine-related compound.
